(19) United States
(12) Patent Application Publication
(10) Pub. No.: US 2024/0274295 A1
(43) Pub. Date: Aug. 15, 2024

(54) METHOD FOR SCREENING FOR RETINOPATHY OF PREMATURITY, APPARATUS FOR SCREENING FOR RETINOPATHY OF PREMATURITY, AND LEARNED MODEL

(71) Applicant: OSAKA UNIVERSITY, Osaka (JP)

(72) Inventors: Yoko FUKUSHIMA, Osaka (JP); Kohji NISHIDA, Osaka (JP); Ryosuke KOJIMA, Kyoto (JP); Yasushi OKUNO, Kyoto (JP)

(21) Appl. No.: 18/566,168

(22) PCT Filed: May 31, 2022
(86) PCT No.: PCT/JP2022/022042

§ 371 Date: Dec. 1, 2023

(30) Foreign Application Priority Data
Jun. 3, 2021 (JP) 2021-093588

(51) Int. Cl. G16H 50/30; G16H 20/00

(52) U.S. Cl. CPC G16H 50/30; G16H 20/00

(57) ABSTRACT

There are provided a highly versatile method for screening for retinopathy of prematurity, a screening apparatus, and a trained model that are capable of accurately predict the progression of retinopathy of prematurity at appropriate timing. There is provided a method for screening for retinopathy of prematurity to predict the progression of retinopathy of prematurity, the method including a treatment determination step of determining whether or not treatment is indicated for retinopathy of prematurity after a predetermined number of days after birth based on premature infant information including postnatal time-series data on weight, height, and vital signs of a premature infant whose gestational age is less than a predetermined week.

### TECHNICAL FIELD

The present disclosure relates to a method for screening for retinopathy of prematurity, a screening apparatus, and a trained model that predict the progression of retinopathy of prematurity, which are used to assist doctors in diagnosing retinopathy of prematurity.

### BACKGROUND ART

Retinopathy of prematurity (ROP) is a major cause of blindness in childhood, and it is estimated that 50,000 people become blind each year worldwide, and this number is expected to increase further in the future. Mostly, disease development is expected to resolve spontaneously, blindness may occur due to intraocular hemorrhage and retinal detachment, especially in very premature infants. Retinal laser photocoagulation is performed as a standard treatment for these infants with severe ROP. Retinal photocoagulation has long been shown to be effective in suppressing the progression of retinopathy of prematurity, but it is not a preventive treatment method as it avoids blindness at the cost of tissue destruction caused by laser burn. Recently, intraocular administration (intravitreal administration) of vascular endothelial growth factor inhibitors (anti-VEGF drugs) has become a new treatment with no retinal damage. Anti-VEGF drugs have the comparative effect on retinal structure with photocoagulation. Therefore, there are concerns about the potential adverse events thereof on systemic development, and thus it is not suitable for preventive treatment. On the other hand, it has been revealed that if treatment is delayed, the disease worsens and photocoagulation becomes ineffective, leading to a sharp increase in the risk of blindness. Therefore, at present, treatment is performed for cases that have reached a certain level of severity in accordance with stage determination based on the international classification and treatment standards based on the results of randomized trials (The Early Treatment for Retinopathy of Prematurity Study, ETROP) in the United States. Frequent fundus examinations and prompt treatment are required to respond to the therapeutic time window in which treatment is effective.

An example of a method for screening for retinopathy of prematurity is the technique described in Patent Literature 1. This screening method detects tryptase, which may be released by degranulation of mast cells, as a marker substance from blood derived from a subject, and determines whether or not treatment for retinopathy of prematurity is necessary.

In addition, a model developed in Sweden (WINROP) and a model reported in the United States (CHOP-ROP model) are known as methods for screening for retinopathy of prematurity. WINROP targets gestational age of 23 weeks or more and less than 32 weeks, and when gestational age, birth weight, and postnatal weight are input every other week, an alarm will be displayed for cases where there is a possibility of deterioration. Similar to WINROP, the CHOP-ROP model evaluates weekly weight gain and helps reduce the number of medical examinations in the low-risk group.

### CITATION LIST

### Patent Literature

Patent Literature 1: Japanese Patent Application Laid-Open No. 2014-208601

### SUMMARY OF INVENTION

### Technical Problem

However, the method described in Patent Literature 1 requires invasive means of blood sampling, which is not practical. Furthermore, even if it is determined that treatment for retinopathy of prematurity is necessary by the method described in Patent Literature 1, there is a possibility that the retinopathy of prematurity will regress naturally, and even if it is determined that treatment for retinopathy of prematurity is not necessary, retinopathy of prematurity may develop after a few days and become more severe. Therefore, frequent fundus examinations are important for predicting the progression of retinopathy of prematurity.

Prediction models have been developed in Sweden and the United States to reduce the number of screenings for non-progressive cases, but both methods track weight gain after birth, and the problem is that the methods have high sensitivity but low specificity. Furthermore, it has been shown that the accuracy is low when the weights of the population are different (premature infants in developing countries, premature infants with a birth weight of less than 1000 g who are at high risk of developing severe disease).

Therefore, there is a need for a highly versatile method for screening for retinopathy of prematurity, a screening apparatus, and a trained model that are capable of accurately predicting the progression of retinopathy of prematurity at appropriate timing.

### Solution to Problem

One aspect of the present disclosure is a method for screening for retinopathy of prematurity to predict the progression of retinopathy of prematurity, the method including a treatment determination step of determining whether or not treatment is indicated for retinopathy of prematurity after a predetermined number of days after birth based on premature infant information including postnatal time-series data on weight, height, and vital signs of a premature infant whose gestational age is less than a predetermined week. In addition, one aspect of the present disclosure is a screening apparatus that predicts progression of retinopathy of prematurity, the apparatus including a treatment determination unit that determines whether or not treatment is indicated for retinopathy of prematurity after a predetermined number of days after birth based on premature infant information including postnatal time-series data on weight, height, and vital signs of a premature infant whose gestational age is less than a predetermined week.

According to the present method or the present apparatus, it is possible to predict the progression of retinopathy of prematurity by determining whether or not treatment is indicated for retinopathy of prematurity by using postnatal time-series data on weight, height, and vital signs. Many factors are intricately related to the progression from the development of retinopathy of prematurity to the time when treatment is indicated, and these factors change over time. In the present configuration, it is possible to provide a method or apparatus that predicts the progression of retinopathy of prematurity with high accuracy by using a plurality of time-series data.

The present method or the present apparatus uses information including immaturity and changes in general condition over time. For example, gestational age at birth is used as an indicator of immaturity, and vital signs such as heart rate, respiration, and blood oxygen concentration, as well as time-series data including weight and height are used as an indicator of general condition after birth. In addition, it is determined whether or not treatment is indicated for retinopathy of prematurity after a predetermined number of days after birth. A specific example to which the present method or the present apparatus is expected to be applicable includes, in a neonatal intensive care unit, a diagnostic aid that is equipped with a program that monitors the vital signs of premature infants over time and displays warning signs for cases where treatment will be soon indicated for retinopathy of prematurity.

According to the present method or the present apparatus, it is possible to predict the progression of retinopathy of prematurity with higher accuracy than existing models (WINROP and CHOP-ROP models). Providing timely and appropriate treatment for retinopathy of prematurity helps reduce the risk of blindness. On the other hand, the shortage of ophthalmologists skilled in accurate stage determination and treatment has become a serious problem worldwide. However, according to the present method or the present apparatus, even those who do not have highly specialized knowledge and experience are able to judge application of treatment and start treatment at an appropriate time. In addition, the stage of retinopathy of prematurity is generally determined by an ophthalmologist by fundus examination using an indirect ophthalmoscope or retinal images obtained with a contact fundus camera, but both methods place a heavy burden on a fragile premature infant, and are associated with the risk of heart rate slowing during medical examinations and imaging. The present method or the present apparatus does not require fundus examination or imaging, but determines whether or not treatment is indicated for a premature infant based on premature infant information including postnatal time-series data on the infant's weight, height, and vital signs, reducing unnecessary medical examinations. In other words, the present method or the present apparatus not only contributes to medical care, but the present method or the present apparatus also contributes to the social economy by significantly reducing medical costs, reduced productivity, and social care costs due to visual impairment.

As described above, it is possible to provide a highly versatile method for screening for retinopathy of prematurity or screening apparatus that are capable of accurately predicting the progression of retinopathy of prematurity at an appropriate timing.

Another aspect of the present method further includes a risk determination step of determining a risk of progression of retinopathy of prematurity based on Type 1 ROP or APROP score calculated at the predetermined number of days after birth, in which the treatment determination step is executed only for the premature infant determined to have the risk of progression in the risk determination step. In addition, another aspect of the present apparatus further includes a risk determination unit that determines a risk of progression of retinopathy of prematurity based on Type 1 ROP or APROP score calculated at the predetermined number of days after birth, in which the treatment determination unit determines whether or not the treatment is indicated only for the premature infant determined to have the risk of progression by the risk determination unit.

Since the present method or the present apparatus determines the risk of progression of retinopathy of prematurity, it is possible to estimate the potential degree of progression of retinopathy of prematurity. Then, since the present method or the present apparatus identifies whether or not treatment is indicated only for premature infants determined to have a risk of progression, it is possible to provide a highly accurate method for screening for retinopathy of prematurity or screening apparatus through a two-step evaluation process.

Another aspect of the present method is that the vital signs are at least one of heart rate, respiration rate, and arterial oxygen saturation of the premature infant.

Since such vital signs can be obtained by an existing monitoring device installed in a neonatal intensive care unit, there is no need to develop a new apparatus and thus it is efficient.

Another aspect of the present method is that the premature infant information includes at least one of gestational age and Apgar score of the premature infant.

As described above, if the gestational age and Apgar score of a premature infant are used as premature infant information in addition to the general condition after birth, it is possible to predict the progression of retinopathy of prematurity with higher accuracy.

One aspect of the present disclosure is a trained model that is operated by a computer, in which the trained model is configured with a decision tree consisting of a plurality of branch points arranged in a tree structure, and into which a feature amount is input, which is computed based on premature infant information including postnatal time-series data on weight, height, and vital signs of a premature infant whose gestational age is less than a predetermined week and who has been treated for retinopathy of prematurity and outputs a score indicating whether or not treatment for retinopathy of prematurity is necessary by summing evaluation values at each of the branch points.

A trained model that has undergone machine learning using a decision tree as in the present configuration is highly versatile and helps accurately predict the progression of retinopathy of prematurity at an appropriate timing. In addition, if the premature infant information is processed and feature amounts are computed to be input into the trained model, it is possible to predict the progression of retinopathy of prematurity with higher accuracy.

One aspect of the present disclosure is a trained model that is operated by a computer, in which the trained model is generated with deep learning including a convolutional neural network, and into which premature infant information is input, which includes postnatal time-series data on weight, height, and vital signs of a premature infant whose gestational age is less than a predetermined week and who has been treated for retinopathy of prematurity and outputs a score indicating whether or not treatment for retinopathy of prematurity is necessary.

A trained model that has undergone deep learning including a convolutional neural network as in the present configuration is highly versatile and helps accurately predict the progression of retinopathy of prematurity at an appropriate timing.

Another aspect of the present model is that the premature infant information is information obtained from the premature infant whose gestational age and number of weeks at the time of treatment are 40 weeks or less in total.

If the gestational age and the number of weeks at the time of treatment is greater than 40 weeks in total, since the risk of progression of retinopathy of prematurity is extremely small, the trained model in the present configuration helps accurately predict the progression of retinopathy of prematurity.

Another aspect of the present model is that the premature infant information is information excluding information obtained from the premature infant who is treated early based on a doctor's judgment.

As described above, by excluding special cases where early treatment is carried out based on the doctor's judgment, it is possible to provide a good trained model.

Another aspect of the present model is that the vital signs are at least one of heart rate, respiration rate, and arterial oxygen saturation of the premature infant.

Since such vital signs are obtained by using an existing monitoring device installed in a neonatal intensive care unit, it is possible to secure a large amount of input data for constructing trained models.

Another aspect of the present model is that the premature infant information includes at least one of gestational age and Apgar score of the premature infant.

As described above, if a trained model is constructed by using the gestational age and Apgar score of a premature infant in addition to the general condition after birth, it is possible to predict the progression of retinopathy of prematurity with higher accuracy.

### BRIEF DESCRIPTION OF DRAWINGS

FIG. 1 is an overall diagram of a system that realizes a screening method according to the present embodiment.

FIG. 2 is a block diagram of a screening apparatus according to the present embodiment.

FIG. 3 is a flow diagram for realizing the screening method according to the present embodiment.

FIG. 4 is an explanatory diagram of the screening method according to the present embodiment.

FIG. 5 is a diagram illustrating a relationship between gestational age and treatment results.

FIG. 6 is a diagram illustrating a relationship between the total number of weeks at the time of treatment or gestational age and the number of weeks at the time of treatment and treatment results.

FIG. 7 is a diagram illustrating an example of a risk determination step.

FIG. 8 is a diagram illustrating the degree of contribution of each feature amount in machine learning.

FIG. 9 is an ROC curve diagram of an example in which a treatment determination step is executed by using a trained model that has undergone machine learning.

FIG. 10 is an AUC diagram of an example in which the treatment determination step is executed by using a trained model that has undergone deep learning.

### DESCRIPTION OF EMBODIMENTS

Hereinafter, an embodiment of a method for screening for retinopathy of prematurity, a screening apparatus, and a trained model according to the present disclosure will be described with reference to drawings. However, the embodiment is not limited to the following embodiment, and various modifications may be made without departing from the gist of the embodiment.

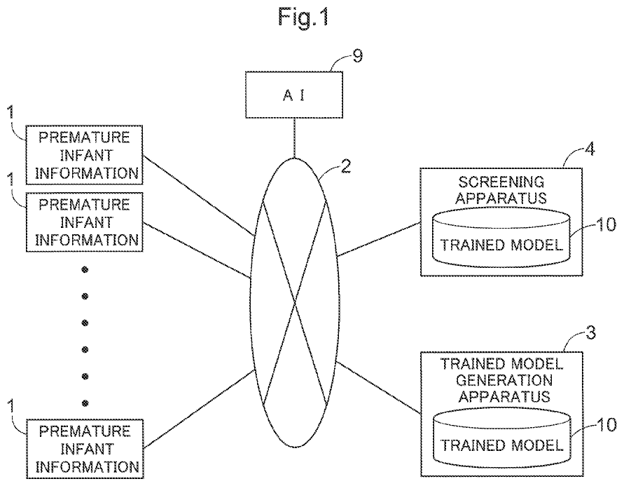

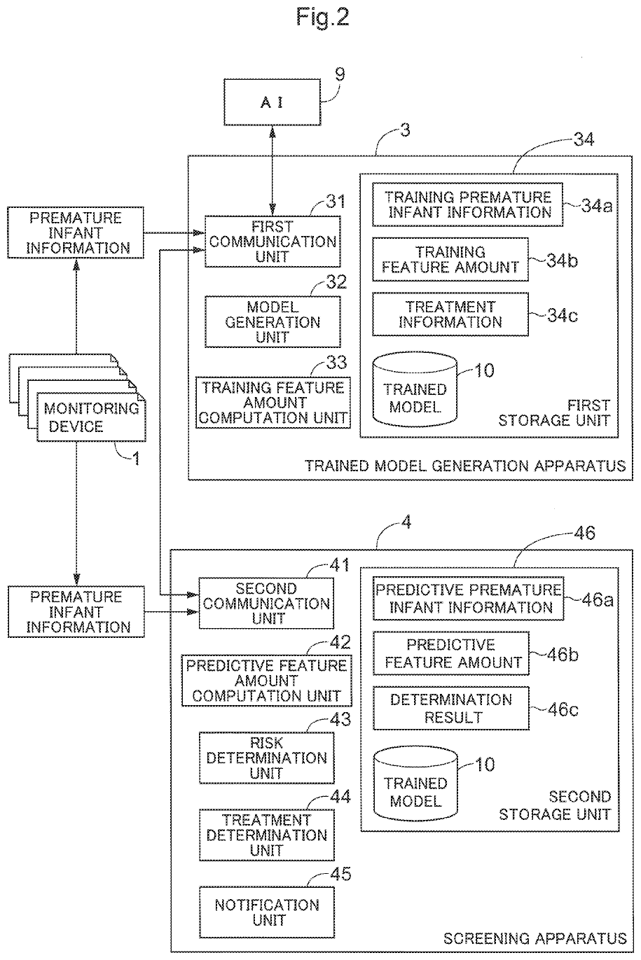

The configuration of a system used in the method for screening for retinopathy of prematurity will be described with reference to FIGS. 1 and 2.

One or a plurality of monitoring devices 1 for acquiring premature infant information including postnatal time-series data on weight, height, and vital signs of a premature infant are connected to an Internet line 2. In addition, a trained model generation apparatus 3, a screening apparatus 4, and an AI 9 (Artificial Intelligence) are connected to the Internet line 2. Here, the AI 9 may be provided on the Internet line 2 or may be provided in the trained model generation apparatus 3. Further, the trained model generation apparatus 3 and the screening apparatus 4 may be the same apparatus, or the screening apparatus 4 may be built in the monitoring device 1, and the respective functions can be used alone or in combination. In addition, the screening apparatus 4 may be used as a monitor apparatus provided in an incubator or a dedicated device in a neonatal intensive care unit, and can be used as various diagnostic aids that predict the progression of retinopathy of prematurity.

The monitoring device 1 is a device that monitors the vital signs of a premature infant over time in a neonatal intensive care unit, and a device that periodically measures the weight and height of the premature infant. The premature infant information of the present embodiment includes the weight, height, and vital signs of a premature infant whose gestational age is less than a predetermined week (for example, 28 weeks). This predetermined week is 36 weeks or less (so-called premature infant), preferably 32 weeks or less, and more preferably 28 weeks or less (so-called very premature infant) (the same applies hereinafter). In the present embodiment, the gestational age at birth is used as an indicator of immaturity, but birth weight may also be used as an indicator. This birth weight is less than 2500 g (so-called low birth weight infant), preferably less than 1500 g (so-called very low birth weight infant), and more preferably less than 1000 g (so-called very much low birth weight infant). The vital signs are at least one of the heart rate, respiration rate, and arterial oxygen saturation of a premature infant, and are obtained at least every minute. This vital signs may include blood pressure and the like as long as the information is vital information of a premature infant. It is preferable that the premature infant information includes at least one of the weight of a premature infant acquired three times a week, the height of the premature infant acquired once a week, the gestational age of the premature infant, and Apgar score at 1 minute and 5 minutes after birth. The frequency of acquisition of premature infant information is not particularly limited, such as every 1 second or every 10 minutes for vital signs, or every day for weight and height. The Apgar score is obtained by evaluating the condition of a newborn immediately after birth with a total score of 10 based on five items: skin color, heart rate, reaction, muscle tension, and respiration.

As illustrated in FIG. 2, the trained model generation apparatus 3 includes a first communication unit 31, a model generation unit 32, a training feature amount computation unit 33, and a first storage unit 34.

The first communication unit 31 is an interface that transmits and receives data to and from the monitoring device 1, the screening apparatus 4, the AI 9, and the like via the Internet line 2. The first communication unit 31 may receive data directly from the monitoring device 1, or may accumulate data acquired by the monitoring device 1 in a server (not illustrated) and receive the accumulated data from the server.

The first storage unit 34 is configured with a non-temporary storage medium such as an HDD or an SSD, or a temporary storage medium such as a RAM, and stores programs and applications executed by a processor. The first storage unit 34 stores training premature infant information 34a of the monitoring device 1 acquired via the first communication unit 31. The training premature infant information 34a includes postnatal time-series data on the weight, height, and vital signs of a premature infant whose gestational age is less than a predetermined week (for example, 28 weeks). Further, the training premature infant information 34a includes the gestational age, Apgar score, and the like of a premature infant. It is preferable that the training premature infant information 34a is information acquired from a premature infant whose gestational age and the number of weeks at the time of treatment are 40 weeks or less in total. Further, it is preferable that the training premature infant information 34a is information excluding the information obtained from the premature infant who is treated early based on a doctor's judgment.

The first storage unit 34 stores treatment information 34c associated with the training premature infant information 34a. This treatment information 34c is classified into no treatment, Type 1 ROP with treatment, and aggressive posterior retinopathy of prematurity (APROP) with treatment. The International Classification of Retinopathy of Prematurity (Classic ROP) includes Type 1 ROP and Type 2 ROP, with Type 1 being suitable for treatment and Type 2 being less than suitable for treatment (no treatment). On the other hand, apart from Classic ROP, a type that becomes worse rapidly is called APROP, which is also suitable for treatment. Type 1 ROP (International Classification) corresponds to Type 1 stage 3 (Ministry of Health and Welfare classification), and APROP (International Classification) corresponds to Type 2 (rush disease). Hereinafter, being suitable for treatment means carrying out treatment out within 72 hours after diagnosis of Type 1 ROP, or carrying out treatment quickly if there are early signs of APROP (the same applies hereinafter). Among Type 1 ROP, ROP suitable for treatment is any of zone 1 ROP with plus disease, zone 1 stage 3 ROP without plus disease, zone 2 stage 3 ROP with plus disease, or APROP. Here, plus disease is one in which dilation and tortuosity of retinal blood vessels are observed, zone 1 is an area within a circle centered on the optic disc and having a radius twice the disc-macula distance, zone 2 is an area within a circle whose radius is from the papilla to the nasal ora serrata, and stage 3 is extraretinal fibrovascular proliferation.

The first storage unit 34 stores a trained model 10. The trained model 10 is a model that is operated by a computer, and is obtained by training with training data of machine learning or deep learning. Further, the first storage unit 34 stores the training feature amount 34b computed by the training feature amount computation unit 33 for machine learning.

Machine learning is configured with a decision tree consisting of a plurality of branch points arranged in a tree structure. This machine learning has a structure in which a feature amount is evaluated at each branch point of a decision tree, and an evaluation value is assigned to each branch point according to the evaluation result. Then, the evaluation values are summed along the branches of the decision tree to obtain progression prediction information for retinopathy of prematurity. Machine learning may be performed by using an ensemble model such as XGBoost, Random Forest, LightGBM, CatBoost, or AdaBoost, in which a plurality of decision trees are provided in association with each other.

Deep learning is executed by AI 9 including a known convolutional neural network (CNN, DCGAN, and the like). The convolutional neural network constructs a deep hierarchical model that mimics a human neural circuit and infers the prediction for the progression of retinopathy of prematurity. This deep learning is configured with a known application provided via the Internet line 2.

The model generation unit 32 includes a processor and generates the trained model 10. The processor includes an ASIC, an FGPA, a CPU, or other hardware for executing an application stored in the first storage unit 34 (the same applies hereinafter). When generating the trained model 10 by machine learning, the model generation unit 32 performs reinforcement learning with the training feature amount 34b as input data and the treatment information 34c (no treatment, Type 1 ROP with treatment, APROP with treatment) as training data to generate the trained model 10. The training feature amount 34b is computed by processing the training premature infant information 34a (postnatal time-series data on weight, height, and vital signs of a premature infant, gestational age and Apgar score of the premature infant), and details will be described later. On the other hand, when generating the trained model 10 by deep learning, the model generation unit 32 performs reinforcement learning with the training premature infant information 34a (postnatal time-series data on weight, height, and vital signs of a premature infant, and the like) as input data and the treatment information 34c (no treatment, Type 1 ROP with treatment, APROP with treatment) as training data to generate the trained model 10.

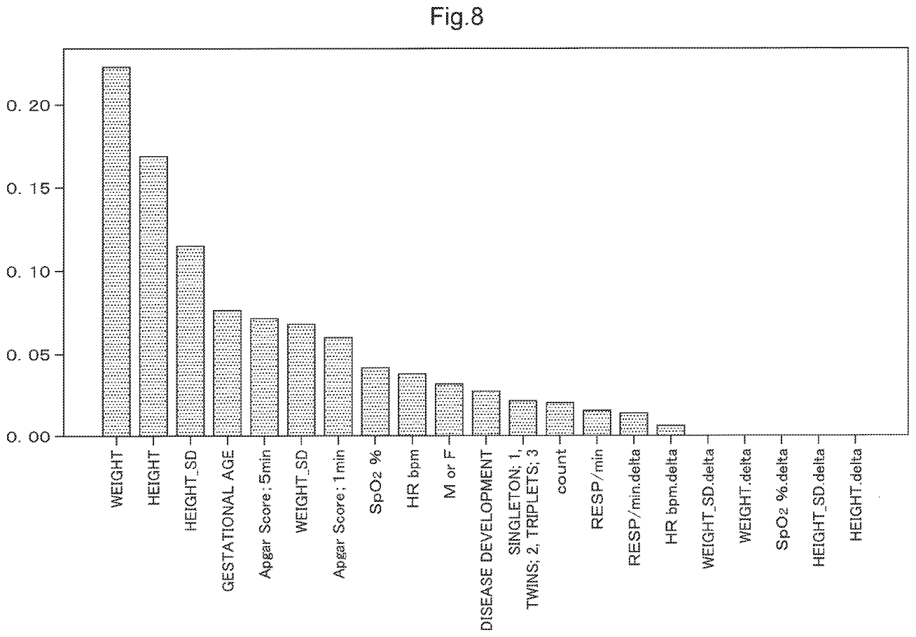

The training feature amount computation unit 33 includes a processor, computes a plurality of training feature amounts 34b from the training premature infant information 34a, inputs the plurality of the computed training feature amounts 34b to the trained model 10 and analyzes, and determines the degree of influence of each feature amount on the treatment information 34c. FIG. 8 illustrates, as an example, the degree of influence of the plurality of training feature amounts 34b on the treatment information 34c. In descending order of the degree of influence of the training feature amount 34b, the training feature amount 34b includes weight, height, height_SD, gestational age, Apgar score 5 minutes after birth (Apgar Score; 5 min), weight_SD, Apgar score 1 minute after birth (Apgar Score; 1 min), arterial oxygen saturation (SpO2%), heart rate (HR bpm), gender (M or F), disease development, birth form (singleton, twins, triplets), count, respiration rate (RESP/min), difference in respiration rate (RESP/min.delta), difference in heart rate (HR bpm.delta), difference in weight_SD, difference in weight, difference in arterial oxygen saturation (SpO2%.delta), difference in height_SD, and difference in height. The training feature amount computation unit 33 may extract a plurality of indicators (for example, 10) in descending order of the degree of influence, and the model generation unit 32 may use the plurality of extracted indicators as the training feature amount 34b.

The weight is the weight of a premature infant after birth obtained three times a week, which is interpolated forward as a daily average value (or hourly average value). The height is the height of a premature infant after birth obtained once a week, which is interpolated forward as a daily average value (or hourly average value). _SD is the magnitude of variation from the average value, that is, the width of the distribution, expressed as a numerical value called SD (standard deviation). Arterial oxygen saturation, heart rate, and respiration rate are obtained by removing zero values from data acquired every minute from the vital signs of a premature infant after birth and interpolated as daily average values (or hourly average values). The disease development is a finding of retinopathy of prematurity (whether or not retinopathy of prematurity has developed) by a doctor that is executed a predetermined number of times after birth. The count is the number of days elapsed from the date of birth converted into a unit time. The difference is obtained by calculating the difference between each measurement of respective parameters and using the difference as a difference feature amount. For the input data of weight, height, and vital signs in the present embodiment, daily average values are used, but any values from a 1 minute average value to a 2-day average value may be used, preferably from a 1 hour average value to a 1 day average value, more preferably an hourly average value or a daily average value (the same applies hereinafter). If the average value of more than 2 days is used as input data, the prediction accuracy will be poor, and if the average value of less than 1 minute is used, the amount of data will be large, which may cause a decrease in computation speed or the inclusion of noise.

Furthermore, when the trained model 10 is generated by deep learning, the training feature amount computation unit 33 is built into the trained model 10. Specifically, weighting is performed inside the trained model 10 (convolution layer) by using time-series feature information, and weighting is performed inside the trained model 10 (attention mechanism) by using a past predicted feature amount. Here, the time-series feature information is the feature amount obtained by arranging the training premature infant information 34a in time series, and the past predicted feature amount is a weighted feature amount extracted as a result of prediction by the trained model 10 itself in order to use weighting of past time-series feature information for current prediction.

The trained model 10 generated in this way outputs a score of whether or not treatment for retinopathy of prematurity is necessary. Similar to the treatment information 34c, in the present embodiment, the necessity of treatment is classified into no treatment, Type 1 ROP with treatment, and APROP with treatment, and the score of the necessity of treatment is expressed by area under the curve (AUC) of each time series for no treatment, Type 1 ROP with treatment, and APROP with treatment. When premature infant information including postnatal time-series data on weight, height, and vital signs of a premature infant whose gestational age is less than a predetermined week (for example, 28 weeks) is input, the trained model 10 is capable of outputting scores for each of no treatment, Type 1 ROP with treatment, and APROP with treatment after a predetermined number of days after birth (for example, 20 days). This predetermined number of days after birth is 1 week or more and 5 weeks or less, preferably 2 weeks or more and 4 weeks or less, and more preferably around 3 weeks (the same applies hereinafter). The score is expressed as a daily or hourly value (for example, time-series AUC). In the present embodiment, the score is calculated every day, but the score calculation interval is every minute to every two days, preferably every hour to every day, more preferably every hour or every day (the same applies hereinafter). If the score calculation interval is more than two days, the prediction accuracy will be poor, and if the score calculation interval is less than 1 minute, the amount of data will be large, which may cause a decrease in computation speed or the inclusion of noise. This score is calculated for each of no treatment, Type 1 ROP with treatment, and APROP with treatment, and if the value of each score indicating treatment is the highest among the scores, a therapeutic time window will be reached soon (treatment is indicated). Here, the therapeutic time window means that in the case of Type 1 ROP with treatment, the treatment is to be carried out within 72 hours, or in the case of APROP with treatment, the treatment is to be carried out quickly. Treatment is selected from any method of retinal photocoagulation, retinal cryocoagulation, intravitreal administration of an anti-VEGF drug, and vitreous surgery, but the treatment is preferably retinal photocoagulation or intravitreal administration of an anti-VEGF drug. Vitreous surgery is carried out when retinal photocoagulation or anti-VEGF drug treatment is insufficiently effective and retinal detachment develops. The trained model 10 may be configured to not only express whether or not treatment is to be carried out in the future (treatment is indicated) as a score, but also output the time to carry out treatment or therapeutic time window.

As illustrated in FIG. 2, the screening apparatus 4 includes a second communication unit 41, a predictive feature amount computation unit 42, a risk determination unit 43, a treatment determination unit 44, a notification unit 45, and a second storage unit 46.

The second communication unit 41 is an interface that transmits and receives data to and from the monitoring device 1, the trained model generation apparatus 3, and the like via the Internet line 2. The second communication unit 41 may receive data directly from the monitoring device 1, or may accumulate data acquired by the monitoring device 1 in a server (not illustrated) and receive the accumulated data from the server.

The second storage unit 46 is configured with a non-temporary storage medium such as an HDD or an SSD, or a temporary storage medium such as a RAM, and stores programs and applications executed by a processor. The second storage unit 46 stores predictive premature infant information 46a of the monitoring device 1 acquired via the second communication unit 41 and the trained model 10 generated by the trained model generation apparatus 3. The predictive premature infant information 46a includes postnatal time-series data on the weight, height, and vital signs of a premature infant whose gestational age is less than a predetermined week (for example, 28 weeks). Further, the predictive premature infant information 46a includes the gestational age, Apgar score, and the like of a premature infant. It is preferable that the predictive premature infant information 46a is information acquired from a premature infant whose gestational age and the number of weeks at the time of treatment are 40 weeks or less in total.

The second storage unit 46 stores a predictive feature amount 46b computed by the predictive feature amount computation unit 42 in order to be input to the trained model 10 that has undergone machine learning by the trained model generation apparatus 3.

The second storage unit 46 stores a determination result 46c output from the trained model 10. This determination result 46c is time-series data classified into no treatment, Type 1 ROP with treatment, and APROP with treatment. The determination result 46c in the present embodiment includes a receiver operating characteristic (ROC) curve for no treatment, Type 1 ROP with treatment, and APROP with treatment. Further, the determination result 46c includes a time-series area under the curve (AUC) computed from this ROC curve.

The predictive feature amount computation unit 42 includes a processor, and computes a plurality of predictive feature amounts 46b from the predictive premature infant information 46a. This predictive feature amount 46b is the same as the training feature amount 34b except for disease development, and in descending order of the degree of influence of the predictive feature amount 46b, the predictive feature amount 46b includes weight, height, height_SD, gestational age, Apgar score 5 minutes after birth (Apgar Score; 5 min), weight_SD, Apgar score 1 minute after birth (Apgar Score; 1 min), arterial oxygen saturation (SpO2%), heart rate (HR bpm), gender (M or F), birth form (singleton, twins, triplets), respiration rate (RESP/min), difference in respiration rate (RESP/min.delta), difference in heart rate (HR bpm.delta), difference in weight_SD, difference in weight, difference in arterial oxygen saturation (SpO2%.delta), difference in height_SD, and difference in height.

The risk determination unit 43 includes a processor, and at a predetermined number of days after birth (for example, 20 days after birth), the trained model 10 into which the predictive premature infant information 46a is input outputs the risk of progression of retinopathy of prematurity. The risk of progression is expressed as a daily or hourly score. This score is calculated for each of no treatment, Type 1 ROP with treatment, and APROP with treatment, and at a predetermined number of days after birth, if the value of each score indicating treatment is higher than a predetermined value among the scores, it is determined that there is a risk of progression. As an example, the risk determination unit 43 extracts a determination indicator having the highest AUC from the time-series AUC in a plurality of determination indicators consisting of no treatment, Type 1 ROP with treatment, and APROP with treatment at a predetermined number of days after birth, and determines that there is a risk of progression if the AUC of Type 1 ROP with treatment or APROP with treatment is higher than a predetermined value (for example, 0.3). This predetermined value is set between 0.1 and 0.8, preferably between 0.2 and 0.6, and more preferably between 0.3 and 0.5.

The treatment determination unit 44 includes a processor, and when the predictive premature infant information 46a including postnatal time-series data on the weight, height, and vital signs of a premature infant whose gestational age is less than a predetermined week (for example, 28 weeks) is input to the trained model 10, the treatment determination unit 44 determines whether or not treatment is indicated for retinopathy of prematurity after a predetermined number of days after birth (for example, 20 days) based on the output value of the trained model 10. It is preferable that the treatment determination unit 44 determines whether or not only premature infants determined to have a risk of progression by the risk determination unit 43 are suitable for treatment. Whether or not treatment is indicated for retinopathy of prematurity is expressed as a daily or hourly score. This score is calculated for each of no treatment, Type 1 ROP with treatment, and APROP with treatment, and if the value of each score indicating treatment is the highest among the scores, it is determined that the treatment determination unit 44 will soon be reached to the therapeutic time window using retinal photocoagulation or the like (the treatment is indicated). As an example, the treatment determination unit 44 extracts a determination indicator having the highest AUC from the time-series AUC in the plurality of determination indicators consisting of no treatment, Type 1 ROP with treatment, and APROP with treatment, and determines that treatment is indicated if the AUC of Type 1 ROP with treatment or APROP with treatment exceeds the AUC of no treatment. As another example, the treatment determination unit 44 determines, from the time-series AUC of the plurality of determination indicators consisting of no treatment, Type 1 ROP with treatment, and APROP with treatment, that treatment is indicated if the AUC of Type 1 ROP with treatment or APROP with treatment exceeds a treatment threshold (for example, 0.8). This treatment threshold is set between 0.5 and 0.9, preferably between 0.6 and 0.9, and more preferably between 0.7 and 0.8.

The notification unit 45 outputs a warning signal when the treatment determination unit 44 determines that treatment is indicated. The notification unit 45 may be configured with a warning lamp, a warning sound, and the like installed in a device that monitors the vital signs of a premature infant over time in a neonatal intensive care unit, or may be configured with a predetermined notification apparatus provided at a nurse station.

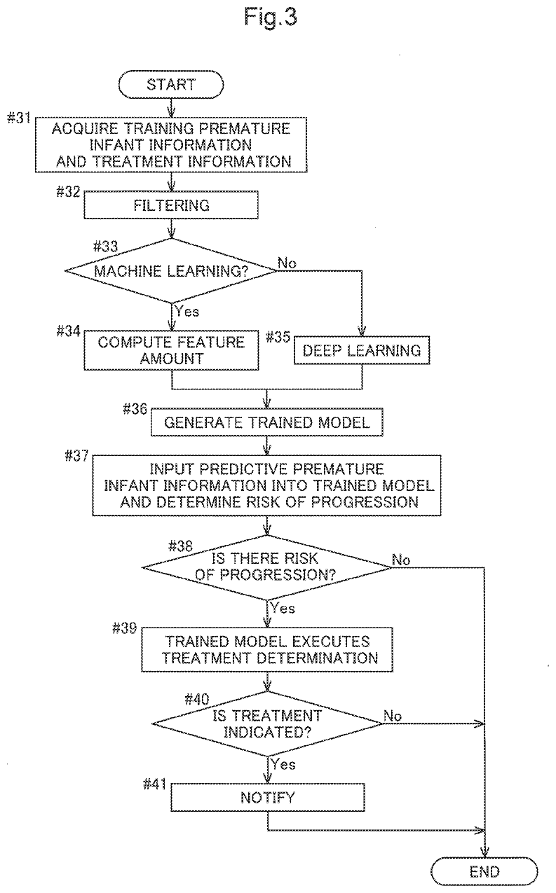

Next, with reference to FIGS. 3 to 10, an example of a method (program) for screening for retinopathy of prematurity, which is executed by a computer to predict the progression of retinopathy of prematurity by using the trained model 10 according to the present embodiment, will be described.

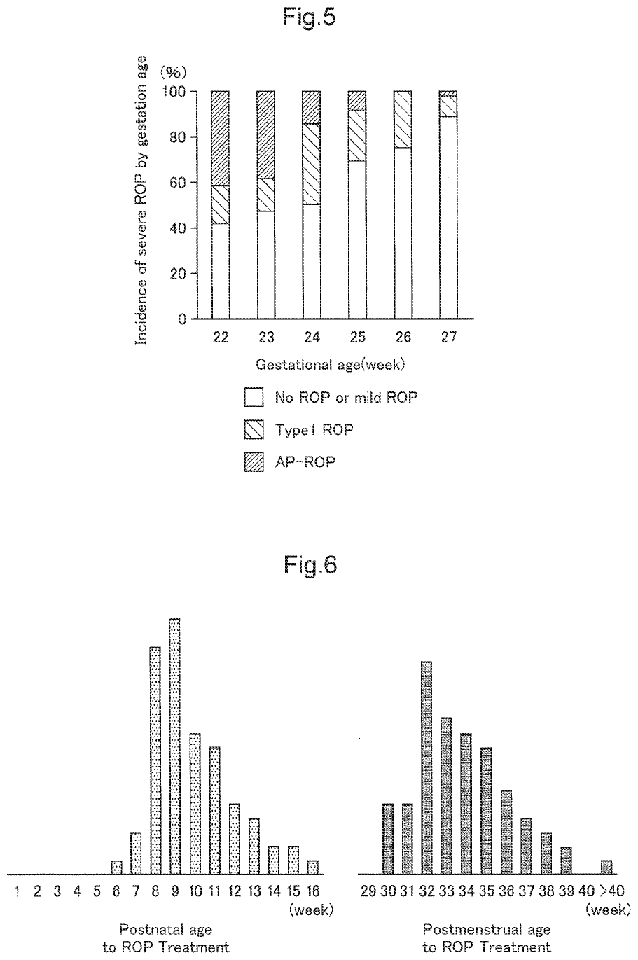

The trained model generation apparatus 3 acquires the training premature infant information 34a and the treatment information 34c over a predetermined period from each monitoring device 1 via the Internet line 2 (#31 in FIG. 3). As illustrated in FIG. 5, according to data on 719 premature infants at Hospital A, the probability of developing retinopathy of prematurity decreases to about 10% when the gestational age reaches 27 weeks. Furthermore, among premature infants whose gestational age is less than 28 weeks, approximately 40% of premature infants with a birth weight of less than 1,000 g are suitable for treatment, and premature infants with a birth weight of less than 1,000 g are at high risk of developing retinopathy of prematurity (APROP) that becomes worse rapidly. Therefore, in the present embodiment, premature infants whose gestational age is less than 28 weeks are targeted for screening. Therefore, the trained model generation apparatus 3 extracts data on a premature infant whose gestational age is less than 28 weeks as the training premature infant information 34a and the treatment information 34c (#32 in FIG. 3, filtering).

As illustrated in the left side of FIG. 6, according to the data of 206 premature infants treated at Hospital A, the time for carrying out the treatment using retinal photocoagulation or the like is the time when number of weeks at the time of treatment is from 6 weeks to 16 weeks after birth. In addition, as illustrated in the right side of FIG. 6, according to the data of 206 premature infants treated at Hospital A, the time for carrying out the treatment using retinal photocoagulation or the like is the time when the sum of the gestational age and the number of weeks at the time of treatment is from 30 weeks to 39 weeks. Therefore, the trained model generation apparatus 3 extracts data on a premature infant whose gestational age and number of weeks at the time of treatment is 40 weeks or less in total, as the training premature infant information 34a and treatment information 34c (#32 in FIG. 3, filtering). The trained model generation apparatus 3 may extract data on a premature infant whose gestational age and number of weeks at the time of treatment is 29 weeks or more and 40 weeks or less in total, as the training premature infant information 34a and the treatment information 34c. Furthermore, the trained model generation apparatus 3 excludes the training premature infant information 34a and the treatment information 34c, which are special cases where early treatment is carried out based on a doctor's judgment (#32 in FIG. 3, filtering). As a result, the training premature infant information 34a in the present example includes time-series data on gestational age, weight, height, respiration rate, heart rate, and arterial oxygen saturation of 206 premature infants treated at Hospital A, Apgar score 5 minutes after birth, Apgar score 1 minute after birth, gender, birth form, count, and disease development (the terms are defined above).

Next, the model generation unit 32 of the trained model generation apparatus 3 performs reinforcement learning with the training premature infant information 34a (postnatal time-series data on weight, height, and vital signs of a premature infant, and the like) as input data and the treatment information 34c (no treatment, Type 1 ROP with treatment, APROP with treatment) as training data to generate the trained model 10 (#33 to #36 in FIG. 3). When the model generation unit 32 performs machine learning (#33 in FIG. 3, Yes), the training feature amount computation unit 33 computes a plurality of training feature amounts 34b from the training premature infant information 34a (#34 in FIG. 3, feature amount computation step). This training feature amount 34b includes gestational age, daily average weight, difference in weight, weight_SD, difference in weight_SD, daily average height, difference in height, height_SD, difference in height_SD, daily average value of respiration rate, difference in respiration rate, daily average value of heart rate, difference in heart rate, daily average value of arterial oxygen saturation, difference in arterial oxygen saturation, Apgar score 5 minutes after birth, Apgar score 1 minute after birth, gender, birth form, count, and disease development (the terms are defined above). As illustrated in FIG. 8, when the plurality of training feature amounts 34b computed by using the training premature infant information 34a of 206 premature infants treated at Hospital A are input into the trained model 10 and analyzed, the degree of influence of each feature amount on the treatment information 34c is determined. Then, the model generation unit 32 performs reinforcement learning with the training feature amount 34b as input data and the treatment information 34c (no treatment, Type 1 ROP with treatment, APROP with treatment) as training data to generate the trained model 10 (#36 in FIG. 3).

On the other hand, when not performing machine learning (#33 in FIG. 3, No), the model generation unit 32 performs deep learning including a convolutional neural network (#35 in FIG. 3). In this deep learning, the model generation unit 32 performs reinforcement learning with the training premature infant information 34a (postnatal time-series data on weight, height, and vital signs of a premature infant, and the like) as input data and the treatment information 34c (no treatment, Type 1 ROP with treatment, APROP with treatment) as training data to generate the trained model 10 (#36 in FIG. 3).

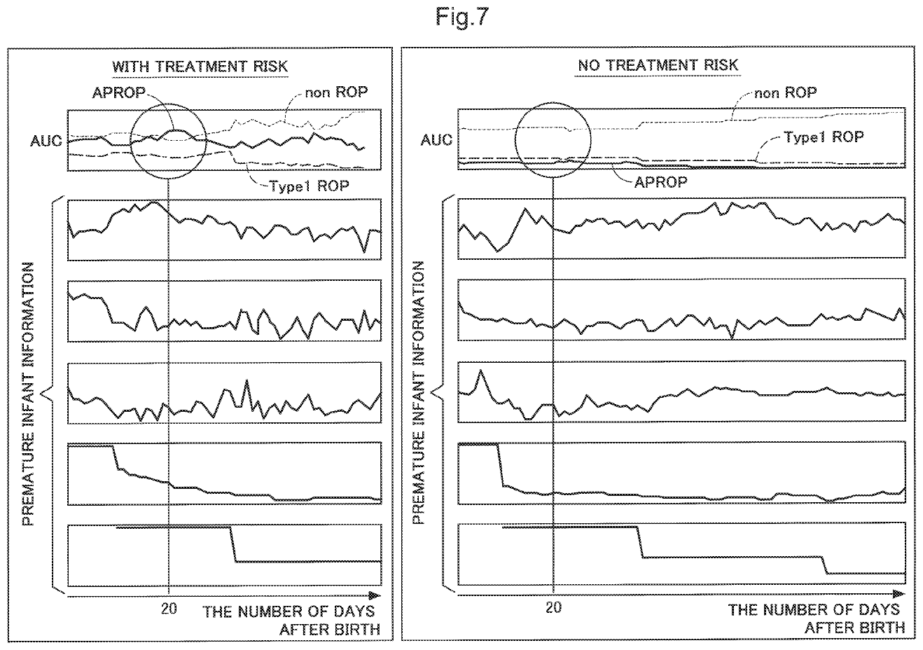

Next, in the screening apparatus 4, at 20 days after birth, the trained model 10 into which the predictive premature infant information 46a is input outputs scores (time-series AUC computed from ROC curve) for each day (see FIG. 7), and the risk determination unit 43 determines a risk of progression of retinopathy of prematurity based on the Type 1 ROP or APROP score (#37 in FIG. 3, risk determination step). This predictive premature infant information 46a includes time-series data on gestational age, weight, height, respiration rate, heart rate and arterial oxygen saturation, Apgar score 5 minutes after birth, Apgar score 1 minute after birth, gender, birth form, and count (the terms are defined above). This score is calculated for each of no treatment, Type 1 ROP with treatment, and APROP with treatment, and if the value of each score indicating treatment is higher than a predetermined value (for example, 0.3) at 20 days after birth, it is determined that there is a risk of progression (#38 in FIG. 3: YES, in the left side of FIG. 7). On the other hand, if the value of each score indicating treatment is less than or equal to the predetermined value, it is determined that there is no risk of progression (#38 in FIG. 3: NO, in the right side of FIG. 7).

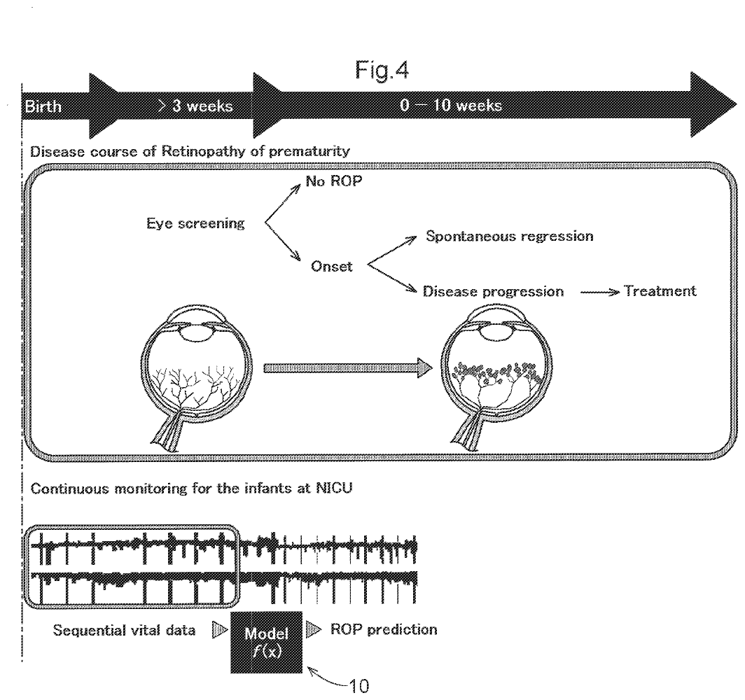

Next, as illustrated in FIG. 4, the predictive premature infant information 46a determined to have a risk of progression is set as onset data (Onset), and the treatment determination unit 44 inputs the predictive premature infant information 46a, which is onset data, into the trained model 10, and determines whether or not treatment is indicated for retinopathy of prematurity after 20 days after birth based on the output value of the trained model 10 (#39 in FIG. 3, treatment determination step). The trained model 10 in the present embodiment is capable of distinguishing between cases that will heal naturally (spontaneous regression) and cases that are suitable for treatment (disease progression) among the predictive premature infant information 46a determined to have a risk of progression. In the case of the trained model 10 that has undergone machine learning, to the trained model 10, the treatment determination unit 44 inputs the predictive feature amount 46b including gestational age, daily average weight, difference in weight, weight_SD, difference in weight_SD, daily average height, difference in height, height_SD, difference in height_SD, daily average value of respiration rate, difference in respiration rate, daily average value of heart rate, difference in heart rate, daily average value of arterial oxygen saturation, difference in arterial oxygen saturation, Apgar score 5 minutes after birth, Apgar score 1 minute after birth, gender, birth form, and count.

When the predictive premature infant information 46a on premature infants determined to have a risk of progression by the risk determination unit 43 is input to the trained model 10, the treatment determination unit 44 determines whether or not treatment is indicated for retinopathy of prematurity after 20 days after birth based on the output value of the trained model 10. Specifically, if the value indicating treatment is the highest among the scores for each of no treatment, Type 1 ROP with treatment, and APROP with treatment, the treatment determination unit 44 determines that the treatment determination unit 44 will soon be reached to the therapeutic time window using retinal photocoagulation or the like (treatment is indicated) (#40 in FIG. 3, Yes), and the notification unit 45 provides notification by a predetermined means (#41 in FIG. 3). In the example illustrated in the left side of FIG. 7, since the score of APROP with treatment (AUC) reached the highest at about 3 weeks after birth, it is determined that treatment is indicated.

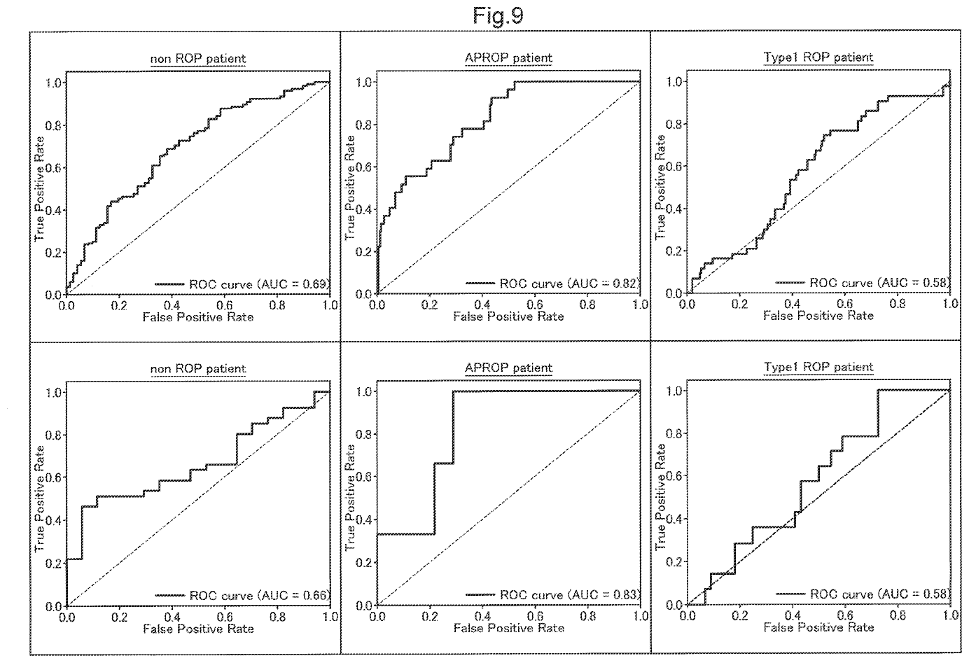

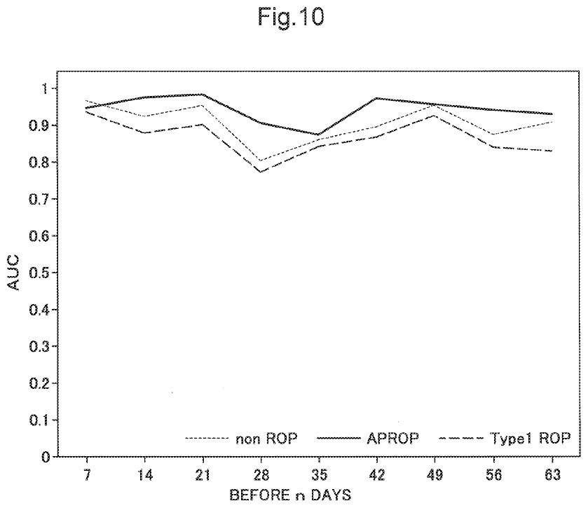

FIG. 9 illustrates the progression prediction performance of retinopathy of prematurity using the trained model 10 that has undergone machine learning with the above-mentioned training feature amount 34b at Hospital A (206 premature infants) as input data, and FIG. 10 illustrates the performance in predicting the progression of retinopathy of prematurity using the trained model 10 that has undergone deep learning with the above-mentioned training premature infant information 34a at Hospital A (206 premature infants) as input data. The verification data illustrated in FIG. 9 was obtained by inputting the above-mentioned predictive feature amount 46b into the trained model 10 and expressing the progression prediction performance of retinopathy of prematurity as a score (time-series AUC computed from the ROC curve) for each of no treatment, Type 1 ROP with treatment, and APROP with treatment. The verification data illustrated in FIG. 10 was obtained by inputting the above-mentioned predictive premature infant information 46a into the trained model 10 and expressing the progression prediction performance of retinopathy of prematurity as a score (time-series AUC computed from the ROC curve) for each of no treatment, Type 1 ROP with treatment, and APROP with treatment.

The upper part of FIG. 9 illustrates the progression prediction performance (ROC curve) on the 20th day after birth when the predictive feature amount 46b of Hospital A (206 premature infants) was input to the trained model 10 that has undergone machine learning with the training feature amount 34b of Hospital A (206 premature infants), and the lower part of FIG. 9 illustrates the progression prediction performance (ROC curve) on the 20th day after birth when the predictive feature amount 46b of Hospital B (59 premature infants) was input to the trained model 10 that has undergone machine learning with the training feature amount 34b of Hospital A (206 premature infants). As illustrated in the upper part of FIG. 9, the area under the ROC curve (AUC) of no treatment was 0.69, the AUC of APROP with treatment was 0.82, and the AUC of Type 1 ROP with treatment was 0.58 at Hospital A. These results demonstrate that the progression of retinopathy of prematurity in advanced cases may be predicted with high accuracy. In addition, as illustrated in the lower part of FIG. 9, the area under the ROC curve (AUC) of no treatment at Hospital B was 0.66, the AUC of APROP with treatment was 0.83, and the AUC of Type 1 ROP with treatment was 0.58, which were almost the same as the progression prediction performance at Hospital A. As a result, the trained model 10 that has undergone machine learning with the training feature amount 34b at Hospital A is a highly versatile model that is capable of predicting the progression of retinopathy of prematurity at Hospital B. FIG. 10 illustrates the progression prediction performance (time-series data of AUC) when the predictive premature infant information 46a of Hospital B (59 premature infants) is input to the trained model 10 that has undergone deep learning with the training premature infant information 34a of Hospital A (206 premature infants), and it is time-series prediction performance calculated backward from the date of treatment or discharge. As illustrated in FIG. 10, since the AUC is 0.8 or more at least 50 days or more before treatment, it can be seen that cases in which retinopathy of prematurity is likely to progress to the point where treatment is indicated may be determined without missing the timing. As a result, the trained model 10 that has undergone deep learning with the training premature infant information 34a of Hospital A is a highly versatile model that is capable of predicting the progression of retinopathy of prematurity at Hospital B. As described above, the present embodiment helps predict the progression of retinopathy of prematurity with higher accuracy than existing models (WINROP and CHOP-ROP models). There have been reports of existing models being applied to various countries, but it has been found that there are significant variations in accuracy depending on countries. This is presumed to be due to variations in the level of newborn management practices. The trained model 10 of the present embodiment may also be used in facilities with different newborn management practices. As a result, even those who do not have highly specialized knowledge and experience are able to judge application for treatment and start treatment at an appropriate time.

The trained model 10 in the present embodiment is highly versatile and is capable of accurately predicting the progression of retinopathy of prematurity at appropriate timing. In addition, if the sum of the gestational age and the number of weeks at the time of treatment is greater than 40 weeks, since the risk of developing retinopathy of prematurity is extremely small, the trained model 10 of the present embodiment, which is trained by using premature infant information whose gestational age and number of weeks at the time of treatment is 40 weeks or less in total, is capable of accurately predicting the progression of retinopathy of prematurity.

Further, it is possible to predict the progression of retinopathy of prematurity by determining whether or not treatment is indicated for retinopathy of prematurity after a predetermined number of days after birth by using the trained model 10 that outputs scores for each of no treatment, Type 1 ROP with treatment, and APROP with treatment. Many factors are intricately related to the progression from the development of retinopathy of prematurity until treatment is indicated, and these factors change over time, but the present embodiment is a method of predicting the progression of retinopathy of prematurity with high accuracy by using a plurality of time-series data. Furthermore, in the present embodiment, since the trained model 10 outputs the risk of progression of retinopathy of prematurity, it is possible to estimate the potential degree of progression of retinopathy of prematurity. In order to identify whether or not treatment is indicated only for premature infants determined to have a risk of progression, there is provided a highly accurate method for screening for retinopathy of prematurity through a two-step evaluation process.

### OTHER EMBODIMENTS

(1) The risk determination step in which the trained model 10 outputs the risk of progression of retinopathy of prematurity may be omitted. Even in this case, it is possible to accurately predict the progression of retinopathy of prematurity by the treatment determination step of determining whether or not treatment is indicated for retinopathy of prematurity after a predetermined number of days after birth by using the trained model 10 that outputs scores for each of no treatment, Type 1 ROP with treatment, and APROP with treatment.

(2) The trained model 10 may be generated by machine learning configured of other than a decision tree or deep learning other than a convolutional neural network. For example, known learning methods such as support vector machine and logistic regression can be used.

(3) It is possible to use other parameters as the premature infant information as long as the information includes postnatal time-series data on weight, height, and vital signs.

### INDUSTRIAL APPLICABILITY

The present disclosure can be used for a method for screening for retinopathy of prematurity, a screening apparatus, and a trained model that predict the progression of retinopathy of prematurity.

### DESCRIPTION OF REFERENCE NUMERALS

4: screening apparatus
10: trained model
34a: training premature infant information (premature infant information)
34b: training feature amount (feature amount)
46a: premature infant information for prediction (premature infant information)

1. A method for screening for retinopathy of prematurity to predict progression of retinopathy of prematurity, the method comprising:
a treatment determination step of determining whether or not treatment is indicated for retinopathy of prematurity after a predetermined number of days after birth based on premature infant information including postnatal time-series data on weight, height, and vital signs of a premature infant whose gestational age is less than a predetermined week.

2. The method for screening for retinopathy of prematurity according to claim 1, further comprising:
a risk determination step of determining a risk of progression of retinopathy of prematurity based on Type 1 ROP or APROP score calculated at the predetermined number of days after birth, wherein
the treatment determination step is executed only for the premature infant determined to have the risk of progression in the risk determination step.

3. The method for screening for retinopathy of prematurity according to claim 1, wherein
the vital signs are at least one of heart rate, respiration rate, and arterial oxygen saturation of the premature infant.

4. The method for screening for retinopathy of prematurity according to claim 1, wherein
the premature infant information includes at least one of gestational age and Apgar score of the premature infant.

5. A screening apparatus that predicts progression of retinopathy of prematurity, the apparatus comprising:
a treatment determination unit that determines whether or not treatment is indicated for retinopathy of prematurity after a predetermined number of days after birth based on premature infant information including postnatal time-series data on weight, height, and vital signs of a premature infant whose gestational age is less than a predetermined week.

6. The screening apparatus according to claim 5, further comprising:
a risk determination unit that determines a risk of progression of retinopathy of prematurity based on Type 1 ROP or APROP score calculated at the predetermined number of days after birth, wherein
the treatment determination unit determines whether or not the treatment is indicated only for the premature infant determined to have the risk of progression by the risk determination unit.

7. A trained model that is operated by a computer, wherein
the trained model is configured with a decision tree consisting of a plurality of branch points arranged in a tree structure, and into which a feature amount is input, which is computed based on premature infant information including postnatal time-series data on weight, height, and vital signs of a premature infant whose gestational age is less than a predetermined week and who has been treated for retinopathy of prematurity and outputs a score indicating whether or not treatment for retinopathy of prematurity is necessary by summing evaluation values at each of the branch points.

8. A trained model that is operated by a computer, wherein
the trained model is generated with deep learning including a convolutional neural network, and into which premature infant information is input, which includes postnatal time-series data on weight, height, and vital signs of a premature infant whose gestational age is less than a predetermined week and who has been treated for retinopathy of prematurity and outputs a score indicating whether or not treatment for retinopathy of prematurity is necessary.

9. The trained model according to claim 7, wherein
the premature infant information is information obtained from the premature infant whose gestational age and number of weeks at the time of treatment are 40 weeks or less in total.

10. The trained model according to claim 7, wherein
the premature infant information is information excluding information obtained from the premature infant who is treated early based on a doctor's judgment.

11. The trained model according to claim 7, wherein
the vital signs are at least one of heart rate, respiration rate, and arterial oxygen saturation of the premature infant.

12. The trained model according to claim 7, wherein
the premature infant information includes at least one of gestational age and Apgar score of the premature infant.

13. The trained model according to claim 8, wherein
the premature infant information is information obtained from the premature infant whose gestational age and number of weeks at the time of treatment are 40 weeks or less in total.

14. The trained model according to claim 8, wherein
the premature infant information is information excluding information obtained from the premature infant who is treated early based on a doctor's judgment.

15. The trained model according to claim 8, wherein
the vital signs are at least one of heart rate, respiration rate, and arterial oxygen saturation of the premature infant.

16. The trained model according to claim 8, wherein
the premature infant information includes at least one of gestational age and Apgar score of the premature infant.
